(19) United States
(12) Patent Application Publication
(10) Pub. No.: US 2014/0031314 A1
(43) Pub. Date: Jan. 30, 2014

(54) METHOD OF PREPARATION OF CHITIN AND ACTIVE PRINCIPLE COMPLEXES AND COMPLEXES OBTAINED

(71) Applicant: Pierfrancesco Morganti, Aprilia (IT)

(72) Inventor: Pierfrancesco Morganti, Aprilia (IT)

(21) Appl. No.: 14/111,886

(22) PCT Filed: Apr. 19, 2012
(86) PCT No.: PCT/IB2012/051961

§ 371 Date: Oct. 15, 2013

(30) Foreign Application Priority Data
Apr. 19, 2011 (IT) RM2011A000199

(51) Int. Cl. A61K 47/36; A61K 8/73

(52) U.S. Cl. CPC A61K 47/36; A61K 8/736; A61K 8/735
USPC 514/54

(57) ABSTRACT

The present invention relates to chitin nanofibrils complexes in association with at least one negatively charged polymer and one or more active ingredients; pharmaceutical or cosmetic compositions comprising such complexes, kit for concomitant or sequential administration comprising said compositions in a form suitable for topical administration and in a form suitable for oral administration, and uses thereof.

### STATE OF THE PRIOR ART

Skin diseases often create both health and social serious effects. Skin is the barrier separating body organs and internal tissues from the outside environment. It is also the body part most exposed to the sight of other individuals. Therefore, it is evident how pathological, or even mere skin aging conditions may cause problems, even social ones, to an individual exhibiting such conditions.

Pathological conditions may be represented both by very serious conditions, such as tumors, and by conditions such as dermatitides, eczemas, bullous diseases, squamous diseases, erhytemas, infections and others, which in turn may be sporadic or chronic. On the other hand, skin aging is an ongoing process associated with progressive changes of the skin manifested in the form of thin lines, wrinkles, tissue prolapsing and irregularities in the pigmentation (skin brown spots or depigmentation). Aging is linked partly to a genetically controlled (intrinsic) process and partly to exposure (extrinsic) to atmospheric and chemical agents (sun, cold, pollutants), besides lifestyle (e.g. smoking, low body mass index), and appearance of menopause. These events, appearing more or less incisively and in times varying between individuals, can induce premature skin aging.

A vast number of pharmaceutical and cosmetic formulations are known which target skin tissue, aiming to treat the diseases, the dysfunctions and/or the damages, even mechanical ones, affecting this organ, or even to preserve it from aging-related consequences or, in particular, to protect it as much as possible from consequences of premature aging.

International Patent Application WO2007/099172, to the same Applicant, describes a mixture of substances comprising melatonin and at least one immunostimulating and/or antioxidant substance, wherein at least one of the constituent substances of the mixture is bound to chitin nanofibrils.

International Patent Application WO2006/048829 describes instead a medical surgical composition comprising chitin nanofibrils as active ingredient, as intradermal cutaneous filler applicable by injection or for prevention of infections.

Also complexes of chitin nanofibrils with hyaluronic acid are described in the same application WO '829 and in other literature, but such complexes are never described as vehicles or carriers for other active ingredients.

The same Patent Application WO2006/048829 provides a method for the preparation of chitin nanofibrils.

Some combinations of chitin nanofibrils with active ingredients are described in the literature; e.g., Morganti et al 2011 describes the melatonin, vitamin E and beta glucan combination as extremely effective for skin aging treatment.

Treatment and prevention of skin diseases, skin damages and skin aging are the subject of continuous research aimed at a constant improvement of the technique and obtained results, anti-age compositions being anyhow compositions which should also meet non-allergenicity, tolerability and non-toxicity requirements, being administered topically, orally, parenterally, intradermal and the like. Therefore, a continuous research exists, aimed at developing novel anti-age compositions and novel administration methods meeting tolerability, non-allergenicity and use safety requirements and, at the same time, providing ever more effective results in the treatment of skin diseases, skin damages and skin aging, both intrinsic and extrinsic, and of symptoms associated thereto.

### SUMMARY OF THE INVENTION

The Authors of the present invention have surprisingly found a novel process of preparation of association complexes of chitin nanofibrils (or derivatives thereof), which is a polymer having a mainly electropositive charge, with polymers having a mainly negative charge capable of entrapping the active ingredients both of natural and synthetic origin. These enriched complexes proved to be particularly effective as vehicles or carriers of active ingredients, in the treatment of skin diseases, in the repairing of damages suffered by skin and/or for the treatment of skin aging. In particular, these complexes proved more effective at incorporating the active ingredients of interest and at reaching the target tissue and exerting their therapeutic and/or cosmetic effect, compared to mixtures or preparations in which the substance having a mainly negative charge has not been used. By substance having a “prevalently negative charge” or substance having a “negative charge” are meant substances whose net charge (prevalent in the substance) is the negative one (e.g., above their isoelectric point proteins have a negative net charge). Moreover, complexes described herein exhibit a higher stability overtime with respect to equivalent preparations in which the negatively charged substance has not been used during the preparation process. The presence of suitable negatively charged polymers during the preparation of the compound containing chitin nanofibrils (herein also referred to as CN) and active ingredients, allows formation of complexes in which the active ingredient is more protected from redox degradation and also proves to be more effective in vivo compared to compounds containing only nanochitin and/or one or more active ingredients of interest normally used both in the pharmaceutical and cosmetic field, or for the manufacturing of bioactive weave and non-weave (or nonwoven) fabrics.

Moreover, the Authors have surprisingly found that a topical administration of formulations comprising the above-indicated complexes concomitantly with an oral administration of the same shows, for the same total dosage through the sole topical or oral administration, a synergistic effect linked to the specific administration mode.

Therefore, object of the invention are chitin nanofibrils complexes in association with at least one negatively charged polymer and one or more active ingredients; pharmaceutical or cosmetic compositions comprising such complexes, kit for concomitant or sequential administration comprising said compositions in a form suitable for topical administration (in which this topical administration may be performed also through biofunctional fabrics) and in a form suitable for oral administration, a use of said complexes or said compositions in a method for the treatment of skin diseases, skin damages, of skin aging and the effects associated thereto, a method for the preparation of said complexes and a method for the preparation of said compositions.

### DETAILED DESCRIPTION OF THE FIGURES

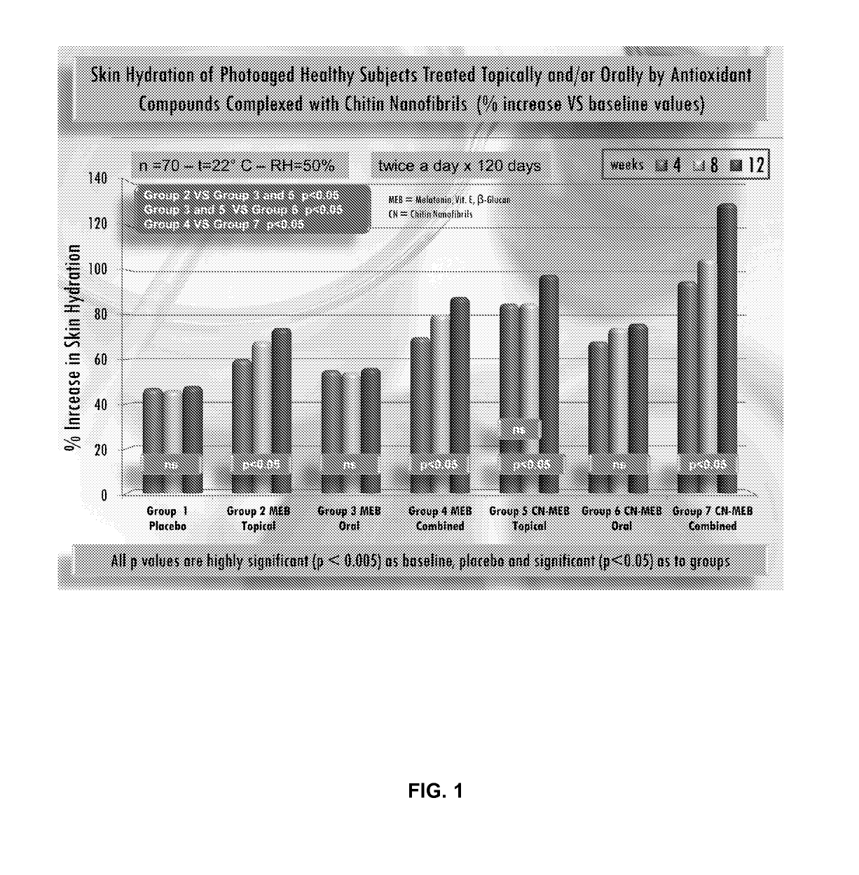

FIG. 1 shows the percent increase of skin hydration overtime (4, 8, 12 weeks) compared to baseline values on healthy subjects exhibiting skin photoaging, treated topically, orally or in a combined oral+topical route with chitin nanofibrils complexes (CN-MEB) according to the invention or with the sole active ingredients not complexed with positively charged chitin nanofibrils (MEB). In particular, in the figures are reported data obtained with MEB represented by a Melatonin+vitamin E+β glucan mixture, while CN-MEB is represented by complexes with chitin nanofibrils according to the invention in which the negatively charged molecule is hyaluronic acid and the active ingredients are the hormone melatonin, the immunostimulant is β glucan and the antioxidant is vitamin E.

CN-MEB or MEB were administered twice a day for a period of up to 120 days, with a total daily dosage equal between the different groups (CN-MEB and MEB) and between the different dosage forms. Therefore, if for oral administration and for topical administration the daily total of active ingredients administered was equal to X, in the case of the combination, the dosage of each individual form of administration was one-half compared to the dosage of the same form of administration alone, therefore ½ of orally administered X+½ of topically administered X each day, for an overall daily total of active ingredients equal to X.

In the figure there may be observed the greater efficacy of CN-MEB with respect to MEB, by comparing the two groups in each of the embodiments, and how the oral+topical administration of CN-MEB be more effective with respect to all other forms of administration, even though the same overall daily amount of active ingredients was administered in all tests performed.

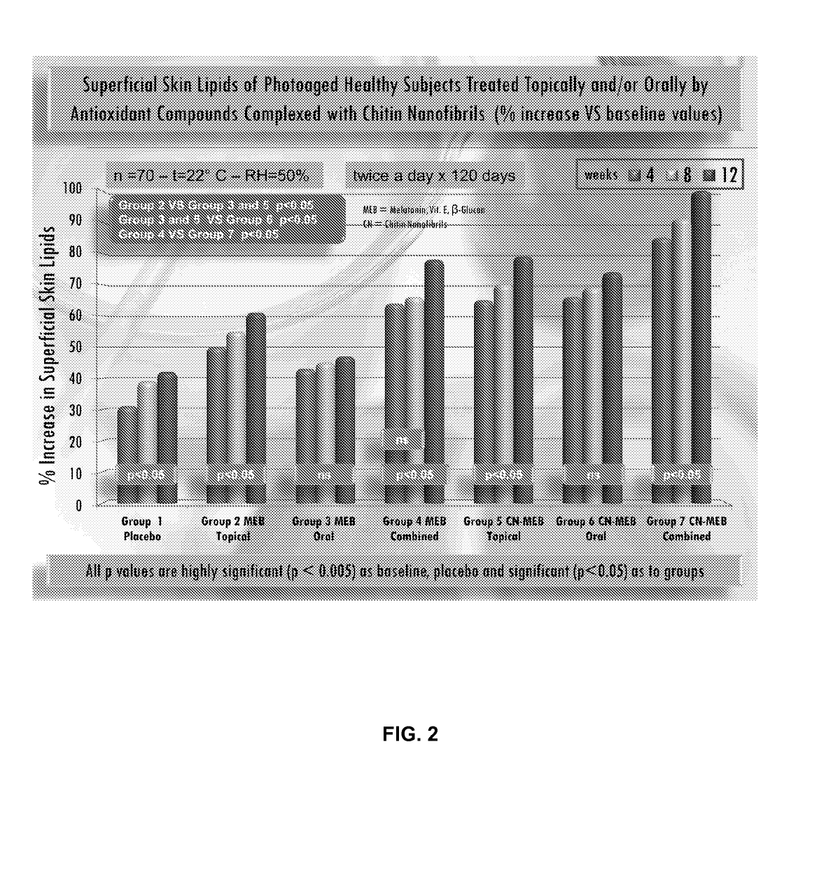

FIG. 2 shows the percent increase of superficial skin lipids overtime (4, 8, 12 weeks) compared to baseline values on healthy subjects exhibiting skin photoaging, treated topically, orally or in a combined oral+topical way with chitin nanofibrils complexes (CN-MEB) according to the invention or with the sole active ingredients not complexed with chitin nanofibrils (MEB). In particular, in the figures are reported data obtained with MEB represented by a mixture of Melatonin+Vitamin E+β glucan, while CN-MEB is represented by complexes with chitin nanofibrils according to the invention, wherein the negatively charged molecule is hyaluronic acid and the active ingredients are hormone melatonin, the immunostimulant is β-glucan and the antioxidant is vitamin E.

CN-MEB or MEB were administered twice a day for a period of up to 120 days, with a total daily dosage equal between the different groups (CN-MEB and MEB) and between the different dosage forms. Therefore, if for oral administration and for topical administration the daily total of active ingredients administered was equal to X, in the case of the combination, the dosage of each individual form of administration was one-half compared to the dosage of the same form of administration alone, therefore ½ of orally administered X+½ of topically administered X each day, for an overall daily total of active ingredients equal to X.

In the figure there may be observed the greater efficacy of CN-MEB with respect to MEB, by comparing the two groups in each of the embodiments, and how the oral+topical administration of CN-MEB be more effective with respect to all other forms of administration, even though the same daily overall amount of active ingredients was being administered in all tests performed.

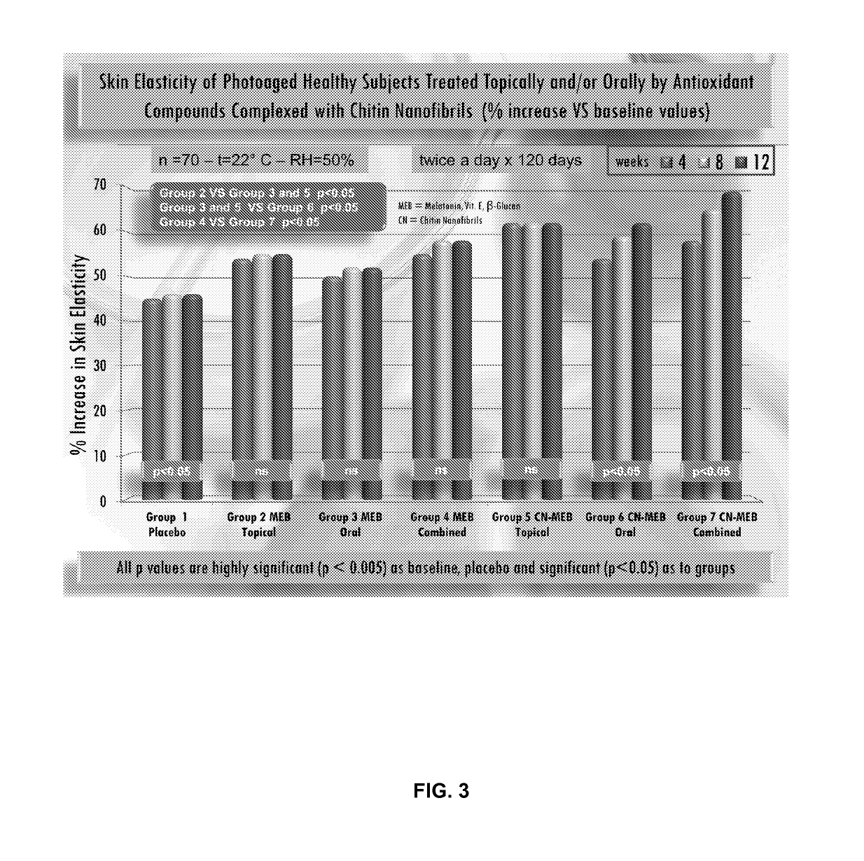

FIG. 3 shows the percent increase of skin elasticity over time (4, 8, 12 weeks) compared to baseline values on healthy subjects exhibiting skin photoaging treated topically, orally or in a combined oral+topical way with chitin nanofibrils complexes (CN-MEB) according to the invention or with the sole active ingredients (MEB) not complexed with chitin nanofibrils (MEB). In particular, in the figures are reported data obtained with MEB represented by a mixture of Melatonin+Vitamin E+β-glucan while CN-MEB is represented by complexes with chitin nanofibrils according to the invention, wherein the negatively charged molecule is hyaluronic acid and the active ingredients are hormone melatonin, the immunostimulant is β-glucan and the antioxidant is vitamin E.

CN-MEB or MEB were administered twice a day for a period of up to 120 days, with a total daily dosage equal between the different groups (CN-MEB and MEB) and between the different dosage forms. Therefore, if for oral administration and for topical administration the daily total of active ingredients administered was equal to X, in the case of the combination, the dosage of each individual form of administration was one-half compared to the dosage of the same form of administration alone, therefore ½ of orally administered X+½ of topically administered X each day, for an overall daily total of active ingredients equal to X.

In the figure there may be observed the greater efficacy of CN-MEB with respect to MEB, by comparing the two groups in each of the embodiments, and how the oral+topical administration of CN-MEB be more effective with respect to all other forms of administration, even though the same daily overall amount of active ingredients was being administered in all tests performed.

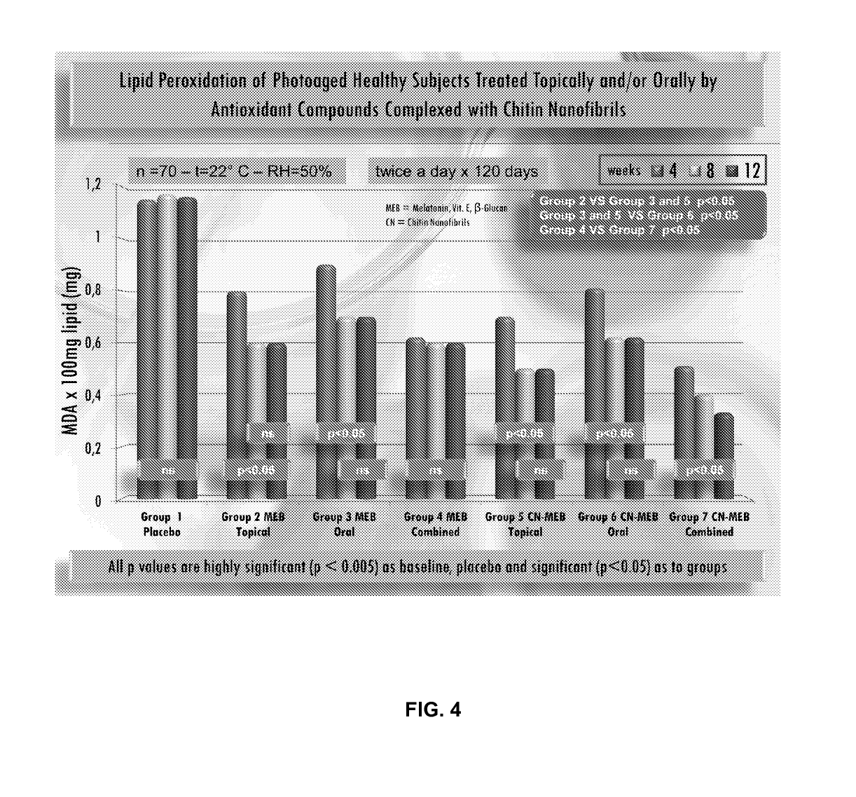

FIG. 4 shows the reduced oxidation of cutaneous skin lipids over time (4, 8, 12 weeks) measured through quantitative checking of MDA (malondialdehyde) compared to baseline values found on healthy subjects who, exhibiting skin photoaging phenomena, were treated topically, orally or in a combined oral+topical way with chitin nanofibrils complexes (CN-MEB) according to the invention or with the sole active ingredients not complexed with chitin nanofibrils (MEB). In particular, in the figures are reported the data obtained with MEB, represented by a mixture of Melatonin+Vitamin E+β-glucan, while CN-MEB is represented by complexes with chitin nanofibrils according to the invention, wherein the negatively charged molecule is hyaluronic acid and the active ingredients are hormone melatonin, the immunostimulant is β-glucan and the antioxidant is vitamin E.

CN-MEB or MEB were administered twice a day for a period of up to 120 days, with a total daily dosage equal between the different groups (CN-MEB and MEB) and between the different dosage forms. Therefore, if for oral administration and for topical administration the daily total of active ingredients administered was equal to X, in the case of the combination, the dosage of each individual form of administration was one-half compared to the dosage of the same form of administration alone, therefore ½ of orally administered X+½ of topically administered X each day, for an overall daily total of active ingredients equal to X.

In the figure there may be observed the greater efficacy of CN-MEB with respect to MEB, by comparing the two groups in each of the embodiments, and how the oral+topical administration of CN-MEB be more effective with respect to all other forms of administration, even though the same daily overall amount of active ingredients was being administered in all tests performed.

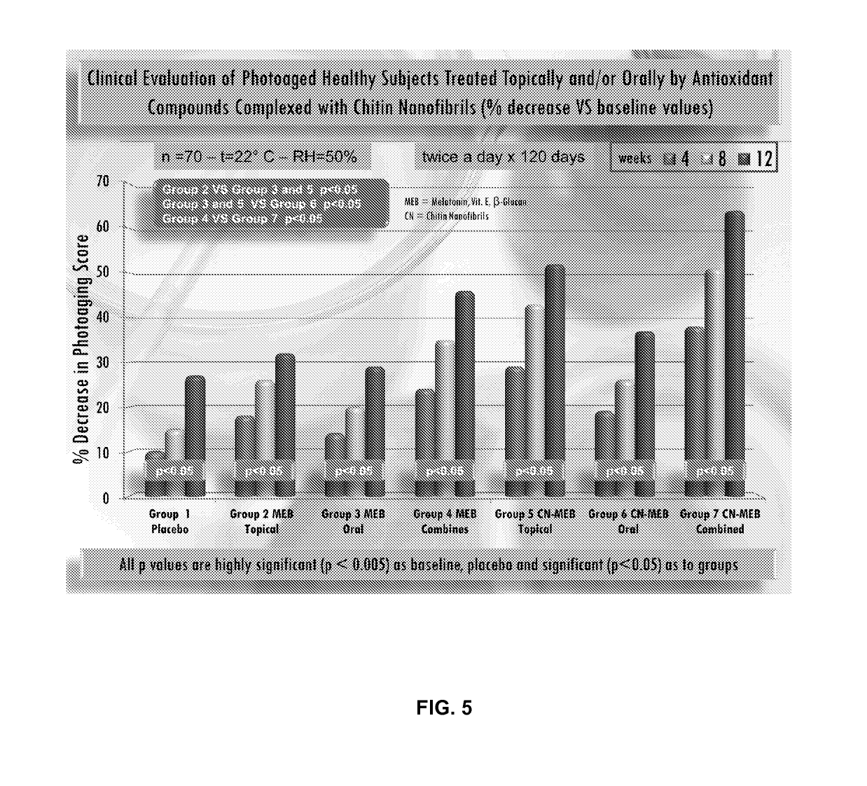

FIG. 5 shows the percent decrease of skin photoaging over time (4, 8, 12 weeks) compared to baseline values on healthy subjects exhibiting skin photoaging treated topically, orally or in a combined oral+topical way with chitin nanofibrils complexes (CN-MEB) according to the invention or with the sole active ingredients not complexed with chitin nanofibrils (MEB). In particular, in the figures are reported data obtained with MEB (a mixture of Melatonin+vitamin E+β-glucan) while CN-MEB is represented by complexes with chitin nanofibrils according to the invention, in which the negatively charged molecule is hyaluronic acid and the active ingredients are hormone melatonin, the immunostimulant is β-glucan and the antioxidant is vitamin E.

CN-MEB or MEB were administered twice a day for a period of up to 120 days, with a total daily dosage equal between the different groups (CN-MEB and MEB) and between the different dosage forms. Therefore, if for oral administration and for topical administration the daily total of active ingredients administered was equal to X, in the case of the combination, the dosage of each individual form of administration was one-half compared to the dosage of the same form of administration alone, therefore ½ of orally administered X+½ of topically administered X each day, for an overall daily total of active ingredients equal to X.

In the figure there may be observed the greater efficacy of CN-MEB with respect to MEB, by comparing the two groups in each of the embodiments, and how the oral+topical administration of CN-MEB be more effective with respect to all other forms of administration, even though the same daily overall amount of active ingredients was being administered in all tests performed.

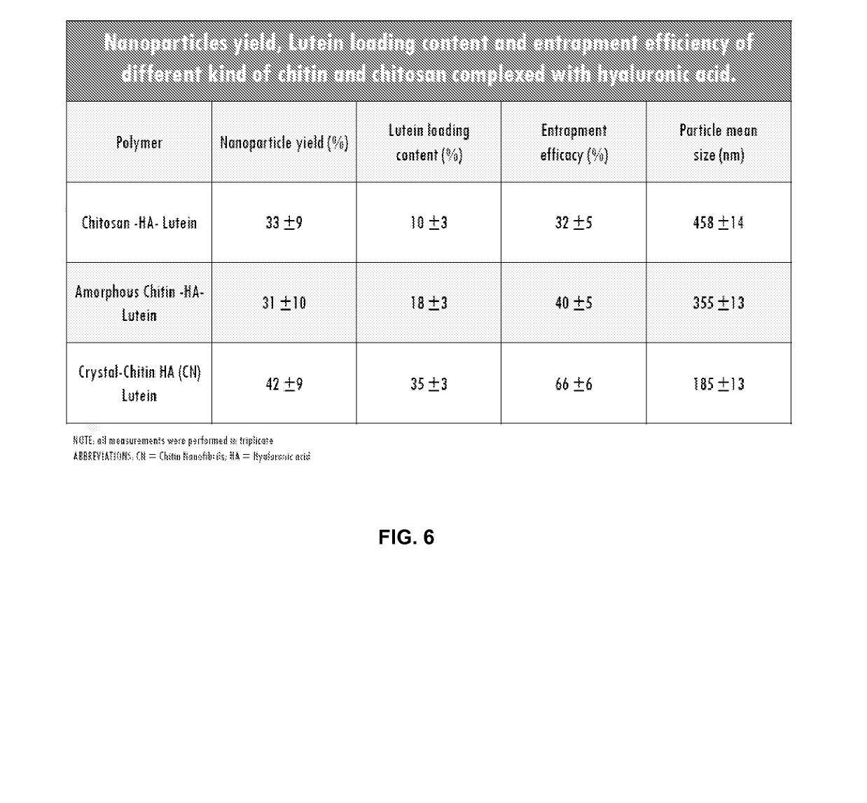

FIG. 6: the table in FIG. 6 reports the formation yields of the complexes between various chitin derivatives and forms (chitosan, amorphous chitin, chitin nanofibrils) and hyaluronic acid; also the lutein loading percents, lutein being an active ingredient, the entrapment percents thereof and the complexes sizes are reported.

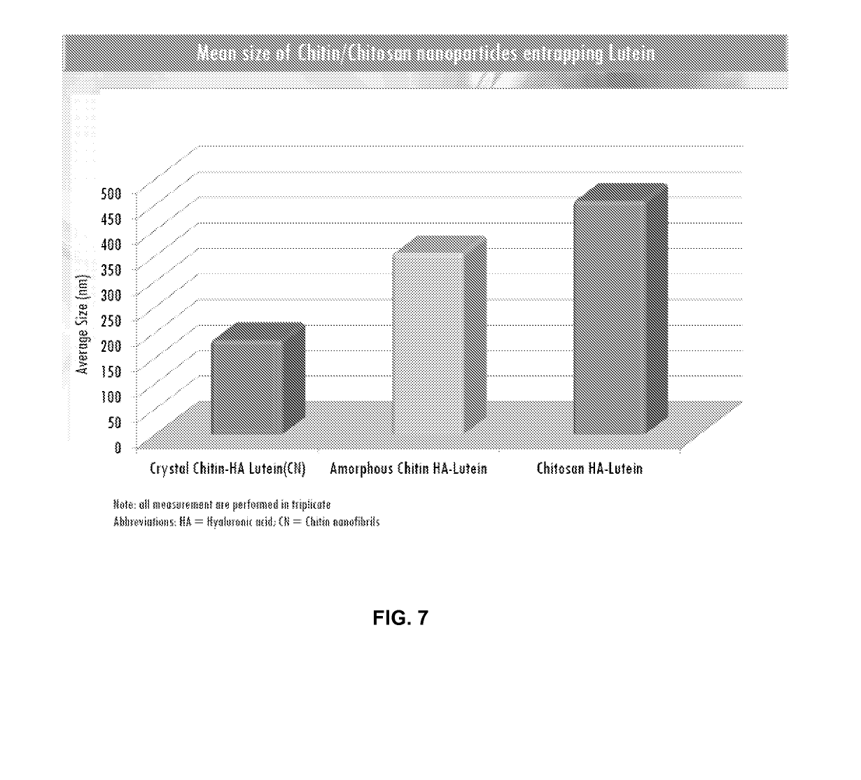

FIG. 7: the block graph in FIG. 7 reports the sizes of the lutein-incorporating complexes, among crystal chitin nanoparticles, amorphous chitin and chitosan with hyaluronic acid.

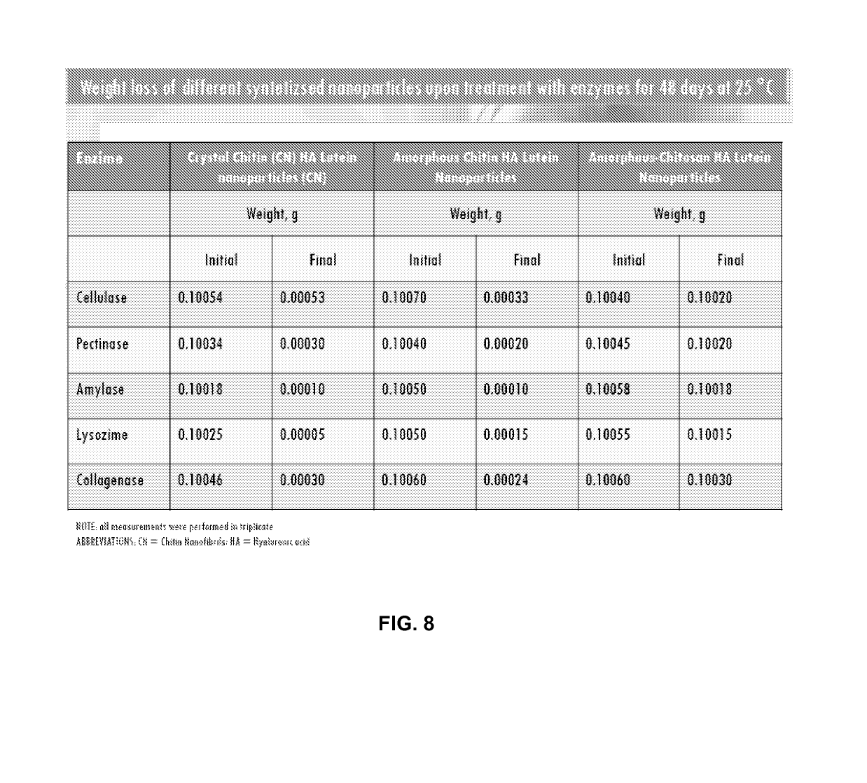

FIG. 8: the table in FIG. 8 reports the biodegradability data of the various complexes between chitin and hyaluronic acid. Biodegradability is measured in terms of weight loss of the mass of the various complexes upon treatment with hydrolytic enzymes for 48 days at 25° C.

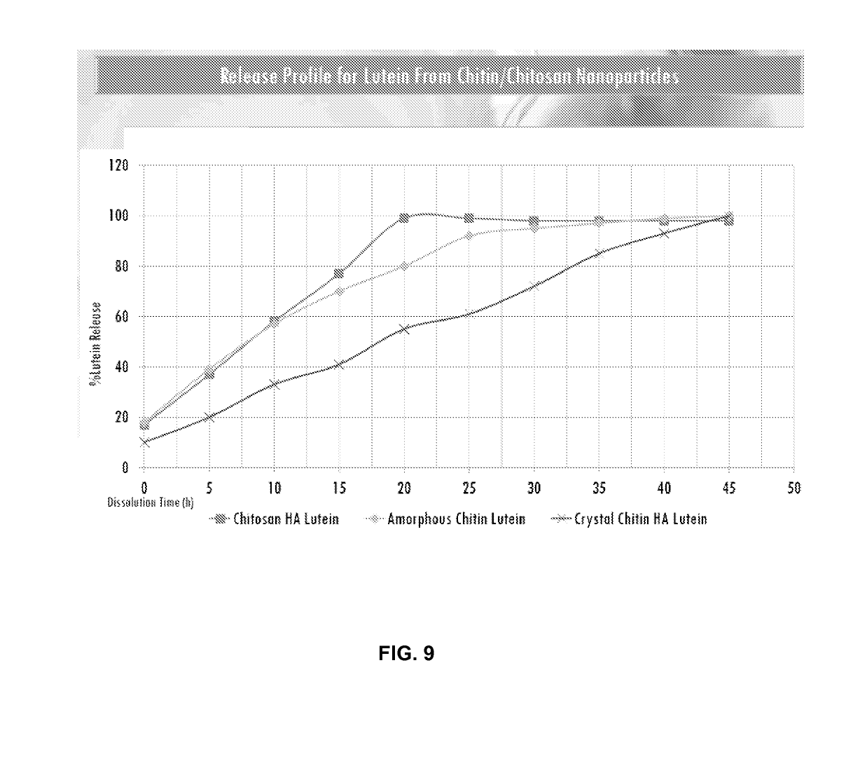

FIG. 9: the graph reported in FIG. 9 illustrates in vitro percent lutein release profiles from complexes of Crystal chitin nanoparticles, amorphous chitin and chitosan with hyaluronic acid. Dark-grey bars represent release.

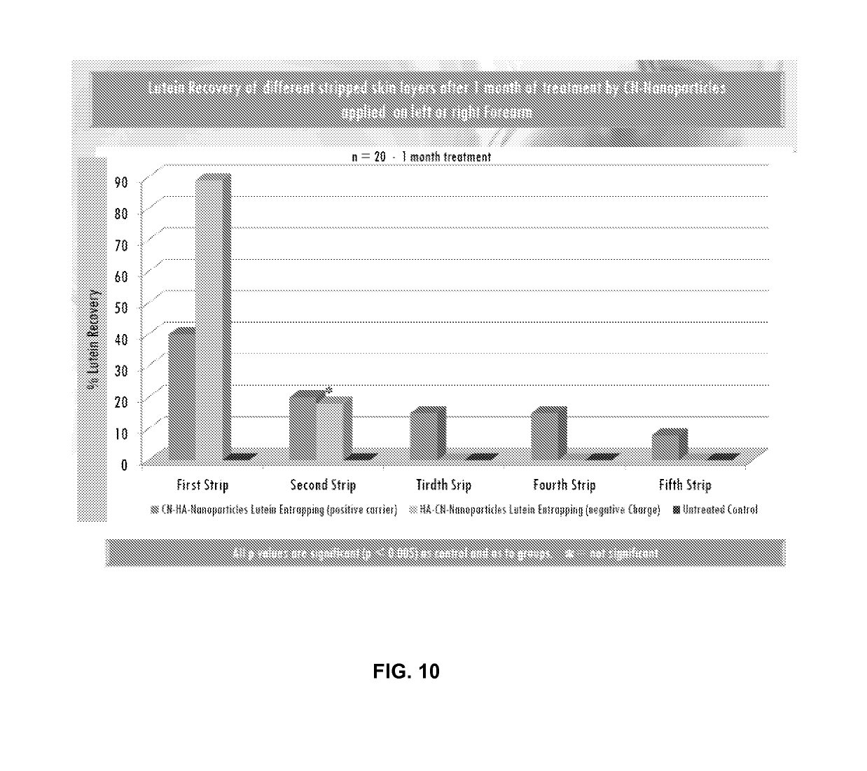

FIG. 10: the block graph of FIG. 10 illustrates in vivo percent lutein release from complexes of Crystal chitin nanoparticles, amorphous chitin and chitosan with hyaluronic acid after 1 month of treatment with such complexes. The test was performed with the stripping method, removing successive layers of skin and analyzing their lutein content. This means that, by going from the “First Strip” to the successive ones, the analyzed skin layers are deeper and deeper. The dark-grey bar represents lutein absorption (recovery) from positively charged complexes obtained by pouring the chitin suspension into the solution/suspension of hyaluronic acid. The light-grey bar represents lutein absorption from negatively charged complexes obtained by pouring the hyaluronic acid suspension/solution into the chitin suspension. As it may be seen, positively charged complexes diffuse homogeneously also in the deepest layers of the skin, while negatively charged complexes accumulate in the most superficial layers of the skin, without penetrating in depth.

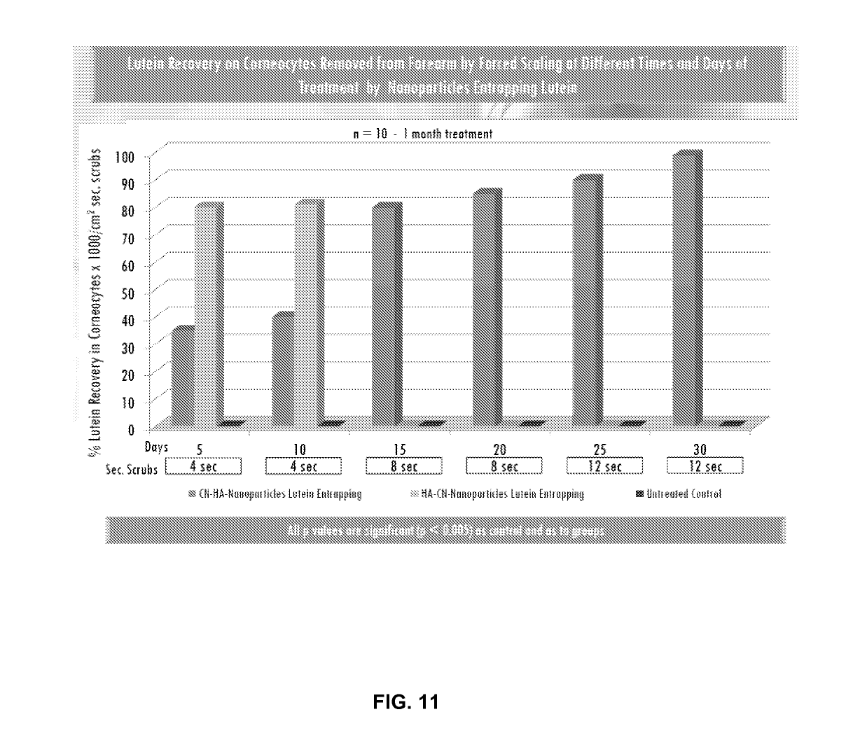

FIG. 11: the block graph of FIG. 11 illustrates the in vivo percent lutein release from compounds of Crystal chitin nanoparticles, amorphous chitin and chitosan with hyaluronic acid. The test was performed by removing skin layers with the “Forced Scaling” method. The results confirm those already reported in FIG. 10.

### DETAILED DESCRIPTION OF THE INVENTION

The authors of the present invention have carried out a method of preparation of chitin nanofibrils complexes in association with at least one negatively charged molecule and one or more active ingredients, which are incorporated very effectively in said complexes. As described more in detail hereinafter, the complexes made with the method of the invention have a greater efficacy in the CN/active ingredients association with respect to the known art, a greater stability of the active ingredients associated therein and a particular effectiveness in medical or therapeutic applications.

Compared to the state of the art, where a bond of active ingredients to chitin nanofibrils is reported (WO2007/099172, p. 19), in which the amount of active ingredient associated to the nanofibrils ranged from 20 to 30% max., the method described herein enables an incorporation of active ingredients (associated to the chitin nanofibrils) even higher than twice what is reported in the literature.

While the literature describes the association between the active ingredient and the nanofibrils by formation of a chemical bond, the method of the invention leads to the production of block copolymer complexes, most likely assembled via Van der Waals forces, inside which the active ingredients are incorporated or “encapsulated”.

Complexes formation via electrostatic interactions is preferred over formation via chemical bonds, as it avoids any toxic side effect often due to the use of organic solvents needed for chemical treatment.

This encapsulation therefore enables CN association with a greater amount of active ingredient, with the three-fold advantage of:
increasing the amount of available active ingredient,
increasing the penetrability of the active ingredient, thanks to the presence of said nanofibrils, and
increasing the stability overtime of the encapsulated active ingredients, with an entailed increase of the half-life of the latter, compared to that of the same active ingredients not encapsulated, and of the storage life of the complexed active ingredients, compared to the same not complexed.

Method of the Invention

The Inventors have observed that, chitin nanofibrils being prevalently positively charged, it is possible to obtain different types of complex associations in the form of microspheres and/or microfibers by mixing the aqueous CN (chitin nanofibrils) suspension with a negatively charged polymer.

In an embodiment of the invention, the method comprises the following steps:
a) preparing a first component of the complex by stirring from 5 min to 2 h a mixture in an aqueous medium of chitin nanofibrils;
b) preparing a second component by suspending in an aqueous medium at least one negatively charged polymer;
c) mixing the first and the second component, thereby obtaining a precipitate and a supernatant. The mixture of the two components can be left to rest, e.g. for about 1 hour or less, so that the complex may stabilize.

The active ingredient(s) or the active ingredients is/are introduced either in component (a), or in component (b), or in both.

The amount of chitin in the first component ranges from 0.1% to 10% (weight/volume), e.g. 0.5%, 1%, 2%, 5% or 8%.

The first component may contain one or more active ingredients, e.g. in concentrations ranging from 0.1 to 10% by weight.

The amount of substance or negatively charged polymer in the second component ranges from 0.1% to 10%, or better from 0.15% to 2%, e.g. 0.5 or 1%. Optionally, the second component comprises one or more active ingredients.

When an active ingredient exhibits hydrophobic features, therefore is liposoluble, the component (a) or (b) in which it is contained will comprise a suitable surfactant which will facilitate the formation of a solution or emulsion or suspension.

The mixing step is usually performed under constant stirring, preferably under high speed. Such a step can take place according to two modes: both by pouring the electronegative polymer solution or suspension in the chitin nanofibrils (CN) suspension, and by pouring the CN suspension into the electronegative polymer preparation. It was observed that the complexes resulting from the two procedures can be different. E.g., when the negatively charged polymer is hyaluronic acid, the first procedure yields a complex having a negative superficial charge, while the second procedure yields a complex having a positive superficial charge.

The precipitate obtained at step (c) may be separated from the supernatant by filtration or centrifugation, and optionally purified and/or subsequently refined to reduce the sizes of the complex to a micro-or nanometric structure.

For instance, according to the suitable techniques known to a person skilled in the art, the separated precipitate obtained at c) or the suspension obtained at c) may be further refined, either by transit into a turbine, or repeated steps (2, 3 or more times) through suitable cylinders under pressure, or suitable homogeneizers or by high-energy ultrasound treatment. Such refining steps will allow to reduce the complexes sizes to a micrometric and/or nanometric scale, preferably below 200 nm.

To this end, suitable mills may be used, such as the colloid one which may be comprised of a frustoconical rotor that rotates, at a very high speed, internally to a stator. The gap between rotor and stator may be adjusted so as to obtain the desired emulsion consistency. The mill also comprises a homogenizer comprised of a coaxial spinneret and a nozzle. The coarse emulsion is laminated and homogenized by crossing the nozzle, whose section can be changed by acting on a micrometer screw connected to the spinneret. The emulsion is kneaded several times and again poured into the mixture, until obtaining the desired homogenizing. For instance, the MK 2000 colloid mill (by Ikausa), especially designed for the production of colloidal mixtures, extremely fine emulsions and suspensions, may be used. Alternatively, numerous other analogous or similar suitable devices are available on the market, e.g. by ART-moderne Labortechnik e. K.; IKA®-Werke GmbH & CO. KG; Probst & Class GmbH & Co. KG; Zoz GmbH; Brogtec Mischtechnik GmbH.

The complexes thus obtained can subsequently be subjected to drying, by spray-drying, or to cryo-spray-drying, and controlled by X-ray diffraction.

Negative Polymers

In the method described herein, the negatively charged polymer or molecule may be selected from the group comprising: hyaluronic acid, collagen, phospholipids and/or synthetic peptides selected from the group comprising polyglucosides, polyphenolic peptides, silicone polymers or oligomers, like e.g. phosphatidylcholine, phosphatidylethanolamine, cyclodextrins, maltoyl- and glucosyl-cyclodextrins, cellulose and derivatives, gelatin, glucose, sucrose, cyclomethicone, derivatives of silanol, alkyldimethicone copolyols, linear and cyclic dimethyl siloxanes, etc.

The preferred negatively charged polymer is hyaluronic acid. Therefore, the complexes obtained by mixing the chitin nanofibrils and hyaluronic acid (HA) represent a specific and preferred embodiment of the invention.

The chitin nanofibrils suitable for carrying out the method described herein are needle-shaped crystallites, also called whiskers, of average length of about 200 nm and a mean diameter lower than 10 nm. Such nanofibrils are prepared, e.g., as described in WO2006/048829. Chitin nanofibrils are chemically different from chitosan and physically different from amorphous chitin and from chitin obtained by electrospinning, which is not produced in the form of a crystalline particle, but of a filament of a length equal to many microns, or even millimeters.

Various active ingredients, liposoluble and/or sensitive active ingredients included, may be encapsulated in the block copolymers of chitin nanofibrils and negative polymer according to the invention. The formation of the complexes thus formed, besides protecting the encapsulated active ingredients, also facilitates their diffusion through the skin.

Any active ingredient compatible with the chitin nanofibrils and with the selected negatively charged substance can be incorporated in the complexes of the invention. Advantageously, given the particular effectiveness on skin of the complexes described herein, active ingredients suitable for treatment of various skin conditions may be used.

In particular, therefore, one or more active ingredients suitable for the treatment of skin diseases and/or for cosmetic treatments of the skin may be selected. For instance, the active ingredients may be selected from those known in the literature for the treatment of fungal, bacterial, viral infections of the skin, dermatitides, eczemas, erythemas, psoriasis, skin tumors, and may be selected also from those known for the treatment of skin aging and of the effects associated thereto.

The active ingredients may be selected between those suitable for treatment of skin damages such as inflammations, lesions, wounds, scars, burns, or for the treatment of skin aging and of the effects associated thereto, and may be selected from hormones, immunostimulants, antioxidants, anti-inflammatory, bactericidal, antifungal, cicatrizing agents, vitamins, oligominerals. For instance, hormones may be selected from the group comprising: melatonin and phytoextrogens, etc.; immunostimulants may be selected from the group comprising: ectoine, beta-glucan, carboxymethyl-betaglucan, zinc gluconate, lactate and picolinate, polyunsaturated fatty acids (PUFA); antioxidants may be selected from the group comprising: carotenoids, polyphenols, lipoic acid, vitamins A, C, E, tocotrienols, coenzyme Q10 and creatine; anti-inflammatory agents may be selected from the group comprising nicotinamide, glycyrrhetic acid, phytosphyngosine, PUFA, corticosteroids, etc; said antifungal agents are selected from the group of zinc pyrithione and pyrithione olamine, ketoconazole, etc.; said bactericidal agents are selected from phytosphyngosine, chlorhexidrine gluconate, glycin, benzoyl peroxide, silver, etc.

Carotenoids may, e.g. be selected from the group comprising: beta-carotene, lutein, zeaxhantin, lycopene, proanthocyanins, flavonoids.

In a specific embodiment of the invention, the active ingredients in the complexes may be selected from melatonin, Vitamin E, beta-carotene, lipoic acid, ectoine, beta-glucan and lutein. In other specific embodiments of the invention the active ingredient is the melatonin, Vitamin E and beta-glucan mixture, or the melatonin, lutein, beta-glucan mixture; or the melatonin, beta-carotene, beta-glucan mixture; or the sole lutein.

In a further embodiment of the invention, the negatively charged molecule will be hyaluronic acid. Therefore, the complexes of chitin nanofibrils and hyaluronic acid, containing each the above-described active ingredients or combinations of active ingredients, represent a preferred embodiment of the invention.

When one or more active ingredients are liposoluble, it may be advantageous to use a surfactant stabilizer in order to facilitate complexes preparation according to the described method.

The active ingredients may therefore be dissolved, emulsified or dispersed, individually or in mixture, in a composition comprising one or more pharmaceutically and/or cosmetically acceptable surfactants. Any commercial surfactant suitable for pharmaceutical use may be used, e.g. fatty acid esters. Also plasticizing and moistening agents may be used, like, e.g., glycerol, oleic acid, lecithin, an oligolactic acid, butylene glycol, ethylene glycol, sorbitol, etc.

The method of the invention enables the preparation of complexes in which the amount of CN-associated active ingredient is substantially greater than that described in the state of the art.

In fact, it was observed that the percent of active ingredient that remains both superficially absorbed, and “entrapped” or encapsulated in the complexes is decidedly higher than those reported in the literature. In particular, the encapsulated percent is higher than 40%, up even to 50%, to 60% and actually even up to about 70% of the active ingredient used for the preparation thereof, with respect to the 20, 30% reported in the known art. The present Inventors compared lutein-encapsulating ability in complexes formed by amorphous chitin-hyaluronic acid (HA), chitosan-HA and Crystal chitin nanoparticles-HA. Incorporation percents and sizes of the particles thus obtained are illustrated in FIGS. 6 and 7. Particle mean size, distribution and zeta potential were determined by a Zetasizer (Nano ZS model Zen 3600, Malvern Instruments, Worchestershire, UK).

Inclusion of active ingredients in the complexes described herein enabled to lengthen both the storage life and the in vivo half-life thereof. In fact, the experimental data reported in the present application indicate how compositions comprising the complexes of the invention show a constant increase of their clinical effectiveness overtime (FIG. 5).

Active ingredient, e.g., lutein, release tests conducted in vitro demonstrated that the release profile from complexes comprising chitin nanofibrils is constant overtime and reaches maximum release (100%) after 40-48 hours, unlike chitosan or amorphous chitin complexes which exhibit maximum release already after 20-25 hours. See FIG. 9.

Likewise, in vivo skin penetration studies demonstrated the efficacy of the complexes of the invention, revealing also an additional characteristic thereof. Precisely, skin penetration is influenced by the external electric charge of the complex. As highlighted in FIG. 10, complexes having an external negative charge will tend to accumulate outside the Stratum Corneum, therefore in the most superficial layers of the skin, whereas positively charged complexes will penetrate more rapidly in the skin, diffusing into the deepest layers. In the case of CN and hyaluronic acid complexes, the total electric charge of the surface depends at least partly on the procedure used for the preparation of the complexes. As already indicated above, complexes obtained by pouring the HA⁽⁻⁾ suspension into the CN⁽⁺⁾ suspension produce negatively charged complexes, while by pouring the CN⁽⁺⁾ suspension into the hyaluronic acid positively charged complexes will be obtained.

Compositions

In order to be administered, the complexes described herein are formulated in a pharmaceutical composition comprising them together with at least one pharmaceutically acceptable excipient and, optionally, one or more among pharmaceutically acceptable adjuvants and/or additives.

Alternatively, the complexes described herein may be formulated in a cosmetic composition comprising them, together with at least one cosmetically acceptable excipient and, optionally, one or more among cosmetically acceptable adjuvants and/or additives.

The pharmaceutical or cosmetic composition may be formulated, e.g., for topical, oral, rectal, vaginal, parenteral, subcutaneous and intradermal administration.

In case the conditions to be treated relate to conditions of the vaginal, rectal or low intestine mucosae, the term “topical administration” may also indicate a vaginal, rectal, nasal or ocular administration. Alternatively, the administration may be mixed, e.g. oral-topical or oral-rectal, even in the combined, concomitant or sequential administration kit mode.

Moreover, the Inventors of the present application have surprisingly discovered that the complexes of the invention proved particularly effective, above all in the cutaneous district, when contemporarily administered orally (by oral route) and topically (by topical route). Data obtained in vivo on healthy individuals who had given their informed consent to the treatment demonstrated that, though the topical route would seem to be more effective at skin level compared to the oral one, for the same total dosage of administered active ingredients, a division of the dosage between contemporary topical and oral administration offers a synergistic effect compared to the sole oral or topical administration (FIGS. 1-5). This result demonstrates not only that the complexes of the invention preserve the activity of the active ingredient even upon oral administration, but also that a combined administration further increases treatment effectiveness.

The various compositions containing the complexes of the present invention are made in liquid, semiliquid/semisolid, solid or spray forms which may be suitable for an oral, topical or parenteral administration and may contain diluents and/or excipients commonly used in the state of the art.

Liquid formulations. Liquid forms for oral administration may include, besides the complexes of the invention, a suitable aqueous or non-aqueous vehicle with buffers, suspending and dispersing agents, emulsifiers, solvents, dyes, aromas and the like.

E.g., they may include water or other solvents, solubilizing and emulsifying agents such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, peanut, corn, wheat germ, olive and sesame oils), glycerol, tetrahydrofurfuryl, polyethylene glycols and sorbitan fatty acid esters and mixtures thereof. Besides inert diluents, the oral compositions may include also adjuvants, such as moistening agents, emulsifying and suspending agents, binding and sweetening, aromatizing and perfuming agents.

Compositions for rectal or vaginal administration may be, e.g., suppositories that can be prepared by mixing the complexes of this invention with suitable non-irritating excipients or carriers such as cocoa butter, polyethylene glycol, or a suppository wax solid at room temperature but liquid at body temperature.

Solid dosage forms for oral administration include hard or soft capsules, lyophilizates, tablets, pastilles, pills, powders and granules. In such solid forms, the complexes of the invention are mixed with at least one inert pharmaceutically acceptable excipient or carrier such as, e.g., sodium citrate or calcium phosphate, and/or fillers or extending agents (such as starches, lactose, sucrose, glucose, mannitol, and silicic acid) binders; (such as, e.g., carboxymethyl cellulose, alginates, gelatin, polyvinylpyrrolidone, sucrose); disintegrating agents (such as agar-agar, calcium carbonate, potato or tapioca starch, alginic acid, certain silicates and sodium carbonate); retarding agents (e.g., paraffin); absorption accelerators (such as quaternary ammonium compounds); wetting agents (such as, e.g., cetyl alcohol and glycerol monostearate); adsorbents (like kaolin and bentonite clay); lubricants (like talc, calcium stearate, magnesium stearate, solid polyethylene glycols, sodium lauryl sulfate) and mixtures thereof.

In case of capsules, tablets and pills, the dosage form may also comprise buffering agents.

The solid compositions as indicated above may also be employed to fill hard or soft gelatin capsules using excipients like lactose or milk sugar, as well as high-molecular weight polyethylene glycols and the like. Solid dosage forms of tablets, sugar-coated pills, capsules, pills, and granules may be prepared with coatings such as enteric coatings and other coating agents known in the technique of pharmaceutical formulations. The formulations described herein may optionally be made so as to release the complexes of the invention only or preferentially in certain parts of the intestinal tract, optionally in a delayed manner.

Moreover, the compositions of the invention may be formulated for topical administration in the form of ointments, pastes, lotions, gels, powders, solutions, sprays, inhalants, ophthalmic ear or nose drops, weave fabrics or nonwoven tissues and natural or synthetic fabrics, films or patches. In case of fabrics and nonwovens, the compositions containing the complexes of the invention may be mixed in suitable doses with other substances normally used to make fibers by electrospinning, i.e., mixtures of chitosan, gelatin, PVA, cellulose, silver, etc. In case of films, the compositions may be mixed with chitosan solutions in suitable solvents which, by evaporation, give rise to formation of elastic and traction-resistant films. The active component(s) is mixed, preferably under sterile conditions, with a pharmaceutically acceptable carrier and any suitable preservative or buffer according to needs.

Transdermal patches may be used to provide a controlled release. Absorption enhancers may also be used, to increase compound flow through the skin. The release rate may be controlled by providing a controlled-rate membrane or dispersing the compound into a polymer matrix or a gel.

Given the peculiar efficacy of the combined administration, the present invention also relates to a kit of parts comprising one or more aliquots of the composition for oral administration as described herein, and one or more aliquots of the composition for topical administration as described herein, intended for use in association.

The aliquots may be subdivided so as to enable administration of the daily dosage divided into one or more unitary doses. The kit may also comprise suitable devices for measuring the formulation for topical and/or oral use, e.g. graduated syringes, measuring cups or the like, and may provide both the oral and the topical formulations in single-dose packages.

The amounts suitable for a single combined oral and topical administration may be packaged in an (oral+topical) blister, so as to facilitate correct administration and dosage of the formulations described herein.

The invention also provides a method of preparation of pharmaceutical or cosmetic compositions or pharmaceutical or cosmetic kit containing the complexes of the invention, comprising a step in which the precipitate of the complexes formed at c) of the above-described method is introduced into a suitable excipient (like, e.g. one of those described above) and the mixture thus obtained is optionally refined.

When necessary, the complexes may also be further washed and/or refined to reduce their sizes from micro to nano and/or sterilized, e.g., by filtration through a bacteria-retaining filter or by association with sterilizing agents.

The present invention also relates to complexes, compositions and kits as described herein, for medical use.

In an embodiment, such medical use may be for the treatment of pathological conditions and/or of alterations of the skin and/or of the cutaneous annexes (nails and hair). These alterations may be, e.g., skin chronoaging, skin photoaging, temporary or definitive skin changes, such as oily or dry skin, keratosis, rosacea, sensitivity to light, skin spots, depigmentation, inflammation; allergic or autoimmune reactions, such as dermatoses and photodermatoses; anomalous cicatrizations, such as skin dystrophy and keloid formations, skin atrophy; loss of skin elasticity, wrinkles, thin lines, stretch marks or cellulitis.

The complexes, the compositions or the kit of the invention may be suitably used also in the treatment of cutaneous pathologies of mucosae or scalp, like e.g. incorrect keratinization, acne, eczema, inflammation and skin or mucosae atrophy, infections, mycoses, bacterioses, lupus erythematosus, atopic dermatisis, psoriasis, eczema, allergic dermatitis, hypersensitivity reactions, burns, eye dryness, cataract, macular degeneration, vaginal dryness and mucosal cancer, skin cancer, cutaneous melanoma, colorectal tumor, vaginal tumor; in scalp treatment, the compositions or the kit of the invention may be used for androgenetic alopecia or in different forms of alopecia, both male and female ones, or in the various forms of hirsutism in a patient in need thereof, wherein said complexes are administered to said patient in therapeutically effective doses.

The active ingredients may be administered in therapeutically effective posologies and dosages, like e.g. those currently used in other preparations for the desired treatment, or with lower dosages, even down to 50% or less than those described in the literature.

By “therapeutically effective dose” is meant a dose which allows the obtainment of the desired therapeutic effect in the treated patient. In the specific case, a therapeutically effective dose will be a dose (administered in one or more unitary dosages over time) leading to a partial or total reduction of the problem of interest in the patient treated.

The therapeutically effective dose may be, as indicated above, administered in one or more unitary dosages by oral and/or topical route (where oral or topical may be replaced by vaginal or rectal, as indicated above) and administration may be associated by oral and topical route, concomitantly or sequentially.

The term “unitary dosage form” “unitary dose” or “unitary dosage” refers to a discrete physical unit suitable for unitary dosages for human or animal subjects, each unitary dose containing a predetermined amount of active material calculated for producing the desired therapeutic effect, in association with a suitable pharmaceutical excipient.

When known active ingredients are used, a technician in the field may use as reference the actual unitary dosage for such active ingredients, and may easily adapt it to the formulations of the present invention.

In the specific embodiment in which formulations are concomitantly administered topically and orally, the overall unitary dosage will be about equivalent to that usable for the sole topical administration or the sole oral administration, and the formulations will therefore be suitably prepared so as to enable administration by topical route of about a unitary dosage means and administration by oral route of a unitary dosage means.

The ratio between what is administered by oral route (orally) and by topical route (topically) may range, e.g., between 10:90 and 90:10 of the two administrations. E.g., the 10, 20, 30, 40, 50, 60, 70, 80 or 90% of the unitary dosage may be administered orally and, respectively, about the 90, 80, 70, 60, 50, 40, 30, 20 or 10% of the unitary dosage topically, so as to administer however concomitantly about the 100% of said unitary dosage.

By way of example, each unitary dose by oral route may comprise about from 1 to 2 mg of each active ingredient, e.g. about 1; 1.1; 1.2; 1.3; 1.4; 1.5; 1.6; 1.7; 1.8; 1.9; 2 mg of each active ingredient individually complexed with about 1.2-1.6 mg, e.g. 1.2; 1.3; 1.4; 1.5; 1.6 mg of chitin nanofibrils/negative polymer.

The topical composition may have known end concentrations of the desired ingredients, e.g. it may contain from about 0.5 micrograms to 500 micrograms/ml, depending on whether it is a composition intended for concentrated intensive applications, or for more extended applications, like e.g. for the face, the body, or localized body parts.

In the present description the terms “comprising”, “comprises” and “comprise” may be optionally limited by the use of the terms “consisting in”, “consists in” and “consist in”, respectively.

The following examples and experiments aim to illustrate the invention and its effects in one of the possible embodiments thereof, and are not to be construed for any reason whatsoever as limitative thereof.

### EXPERIMENTAL SECTION

### Example 1

### Preparation and Characterization of Lutein-Containing CN-HA Complexes

Complexes Preparation

Operating under high speed and constant stirring, block-polymer based nanoparticles were obtained by dropping the acidic suspension of the mycrocrystalline CN into a stabilized suspension of HA and vice versa, utilizing a syringe with a 30 gauge needle. The HA water suspension contained a stabilizer hydrophilic surfactant, while the crystalline-CN suspension contained both a lipophilic stabilizer and lutein as active ingredient. In this way, a satisfactory stable emulsifying system was obtained. Under this conditions, CN undergoes ionic gelation and precipitates in the form of lamellar microparticles entrapping the active ingredient (i.e., lutein). The microparticles mixture, purified by centrifugation, resuspended in demineralised water and treated by high-pressure homogenizer or high-power ultrasounds, was atomized in a stream of hot air, with formation of disaggregated particles.

Characterization

Amorphous Chitin/HA, Chitosan/HA, and Crystal Chitin nanoparticles/HA complexes were respectively used to obtain lutein encapsulation. Then, there were measured: release of amount of lutein from the obtained nanoparticles, particle mean size, zeta potential, encapsulation efficiency and storage stability of the compounds. The main characteristics of the nanocompounds are reported in FIGS. 6 and 7. Lutein loading content for all polymeric complexes varied between 10% and 35%, while encapsulated lutein content was very high. CN-HA-lutein complexes had the highest encapsulation efficiency, i.e. about 66%, while Amorphous CN-HA-lutein had about 40% and Chitosan-HA-lutein ones about 32%. Lutein loading content and entrapment efficiency values were quite satisfactory for all the compounds used, considering that lutein, as an oil soluble ingredient, is rather difficult to be encapsulated into these hydrophilic polymers. As shown in FIGS. 6 and 7 the better entrapment efficiency of lutein in CN-HA complexes may be due to CN crystallinity. Particle mean size, width distribution and zeta potential were determined by a Zetasizer (Nano ZS model Zen 3600, Malvern Instruments, Worchestershire, UK). Each sample was dried by Büchi Mini B-190 spray drier (Flawil, Switzerland), suspended in distilled water, sonicated for 5 minutes and read at a 173° angle in respect to the incident light beam. Zeta potential measurements were performed on the same samples prepared for size analysis. Zeta limits ranged from 120 to 120 mV. Each reported value was the average of 3 measurements. A Scanning Electron Microscope (SEM/EDY, Philips XL30) was used to examine the morphology of the nanoparticles and also to physically measure their relative sizes. The mean nanoparticle diameter for all the polymeric complexes obtained varied from a minimum of 185 nm for the nanocrystal-chitin (CN) complexed with HA, to 485 nm of chitosan-HA complex (FIGS. 6 and 7).

In Vitro Release Study

By following the method of Karavelidis et al (Karavelidis V, Karavas E, Giliopoulos D, Papadimitriou S, Bikiaris D (2011) Evaluating the effects of crystallinity in new biocompatible polyester nanocarriers on drugs release behaviour. Intern. J. Nanomedicine 6: 3021-3032), the rate of lutein release from nanocomplexes was measured in a dissolution apparatus (Distek 2100B) equipped with an autosampler using the paddle method (USP II). Each dissolution vessel was loaded with a quantity of nanoparticles corresponding to 1.5 ng of lutein. The test was performed at 37° C.+/−1° C. with a rotation speed of 100 rpm. The dissolution medium was 500 ml of phosphate buffer solution (pH 7.4) containing a stabilizer. At predetermined time intervals, samples of 5 ml were withdrawn from the dissolution medium. The samples were filtered through 45 μm ultrahigh molecular weight polyethylene filters, and assayed using HPLC with a UV reader at 490 nm. All measurements, performed in triplicate are reported on FIG. 9. As shown from FIG. 9, the lutein release profile from nanoparticles (nanocomplexes) of chitosan and amorphous chitin reaches its maximum value (from 80 to 98%) during the early stages (up to 20 hours), while complexes of nano crystal-chitin induce a continuous and quite constant release of lutein, reaching the maximum value after up to about 45-48 hours.

In Vivo Skin Penetration Test

Transcutaneous penetration was controlled on 20 volunteer subjects, who had given their informed consent, by the stripping method according to Miyajima M, et al. (1999), (Effect of polymer/basic drug interactions on the two diffusion-controlled release from a poly (-lactic acid) matrix.” J. Control Release 61: 295-304) (D-squame®, Cu Derm Co., Dallas, Tex.). On other 10 volunteers the scrub technique was applied, previously modified and used by our group (Morganti P, Randazzo S D, and Bruno C (1996) Alpha Hydroxy acids in the cosmetic treatment of photo-induced skin ageing J. Appl. Cosmetol. 14: 1-8). The Stratum Corneum turnover, and consequently the different stripped skin layers, were previously achieved by the use of dansyl chloride fluorescence method (Jansen L H, Hoiyo-Tomoko MT and Kligman A M (1974) Improved fluorescent staining technique for estimating turnover of the human Stratum Corneum. B.J. Dermatol. 90: 9-12). All subjects were treated in a random manner for 1 month, applying the nanoemulsion containing CN-HA nanoparticles (positively charged) or HA-CN nanoparticles (negatively charged). The nanoemulsions were applied respectively on the right or left forearm by means of a glass spatula (2 g/100 cm2). After accurate cleansing performed 30 minutes after application of product, the treated area was subjected to the various strippings. All strippings were performed sequentially, operating always in the same way and by means of the same operator, so as to obtain a homogeneous removal of the SC. 5 strippings out of 10, performed both in the treated and the untreated area, were analytically assessed to check the concentration of lutein present.

All the nanoparticles, produced always by the same methodology, contained lutein used always at the same concentration. Lutein concentration, extracted from the skin by use of organic solvents (acetone/alcohol 1:1) and obtained by stripping or scrub, was determined by use of a spectrophotometer at a wavelength of 490 nm. The obtained results are reported on FIGS. 10 and 11. It has to be underlined how the use of our experimental method allowed us to produce two different typologies of nanoparticles: positively charged on their external surface (1^{st} case), when CN was poured dropwise into the HA suspension, or negatively charged when HA was poured into the CN suspension. Moreover, the size of the drop will also determine the end size of the nanoparticle. Thus, independently of the method used and as evident in FIGS. 10 and 11, by the use of nanoparticles positively charged (1^{st} case) it was possible to detect how lutein were always present in a greater amount at the first 5 strippings of the SC (2^{nd} stripping=about 20 layers) which represent its deepest portion. In contrast, the negatively charged nanoparticles were found on the outermost surface of the SC (90% on first stripping), it also negatively charged. Considering the intensity of the stripping and the different times of treatment performed with the emulsions containing the nanofibrils, it is evident how the amount of lutein found at the corneocyte level be higher and present at increasing depth, in dependence of the days of treatment (4 sec v 12 sec). More forced scrubs and repeated strippings enabled to control also corneocytes present at the boundary with the active keratinocytes. This phenomenon is more evident with the use of positively charged CN-HA nanoparticles. On the contrary, negatively-charged nanoparticles are positioned only in the most superficial layers of the Corneum. Thus, 100% lutein was found at a very superficial level or in the deepest SC layers, depending on the electrical charge and on the number of strippings and scrubs performed, but however always outside derm. This different activity carried out by nanoparticles, depending on their superficial electrical charge, demonstrates how positive electrical charges be capable of disturbing the even arrangement of lipid lamellae and corneocytes. It seems that these charges are capable of causing the forming of actual penetration tunnels, similarly to what takes place, e.g., with ultrasound use. Thus, both nanoparticle penetration and the release of active ingredients bound or entrapped into the same nanoparticles would be fostered. In conclusion, it seems possible to produce nanostructured carriers comprised of nanoparticles having a positive or negative charge and of different size, capable not only of binding biologically active ingredients, but of selectively releasing them at the level of the different layers of the skin at different times. These innovative nanoparticles, based on the use of CN, seem capable of temporarily changing the physical properties of SC, enhancing the diffusivity of active compounds, and with no need to use more or less invasive techniques.

Biodegradability

The higher or lower degradability of the different nanoparticles was assessed by measuring their weight variation after having subjected them to the hydrolytic action of different enzymes, such as cellulase, pectinase, amylase and collagenase. The results obtained are reported on FIG. 8. As evident from obtained data, all nanoparticles were indiscriminately hydrolyzed by all enzymes used. These results highlight how the nanoparticles based on CN use and produced with our technology be readily and totally degraded by enzymes normally present both in humans and in the surrounding environment: For these characteristics, they could be considered and used as eco- and bio-compatible carriers for standard pharmaceutical or cosmetic or biomedical use.

In Vitro Citotoxicity of CN and CN-Complexes

Cytotoxicity studies were performed on ex vivo cultures of keratinocytes and fibroblasts collected from volunteer subjects, and highlighted how these nanoparticles be completely free from any cytotoxic effect, and on the contrary be used by said cells as a medium for their growth.

### Example 2

### Assessment of Cutaneous Effects of CN-HA Complexes Containing Melatonin, Vitamin E and β-glucan

In the following exemplary experiments, nanochitin complexes were made, comprising, as negatively charged molecule, hyaluronic acid and, as active ingredients, melatonin, Vitamin E and beta-glucan. Complexes were formulated in compositions for topical administration and for oral administration, and were assayed in vivo on healthy patients exhibiting signs of skin aging. Compositions were clinically assayed for their effect on skin hydration, on the presence of surface lipids on the skin, on skin lipid peroxidation, and on skin elasticity.

Compositions were administered in separate groups topically, orally and in a combined topical+oral route. The effects of the different administration routes were compared thereamong and with respect to a control group treated with the sole placebo. Also, complexes efficacy was compared to that of a mixture of the same active ingredients not complexed with chitin nanofibrils according to the invention.

The assays reported below show how the complexes of the invention increase in a statistically significant way the efficacy of active ingredients and improve their penetration through skin. The greater efficacy and the improved penetration ability are to be ascribed to the structure of the complexes described herein and therefore are transferable by analogy to active ingredients, formulated in the complexes of the invention, different from those exemplified herein.

Materials and Methods

By following a protocol approved by the appropriate ethics committees and after informed consent a 12 week clinical trial was performed on seventy healthy woman volunteers (age 22-45 years).

The effects of (oral and topical) compositions containing chitin nanofibrils complexes and hyaluronic acid comprising melatonin, vitamin E and β-glucan, (denoted by CN-MEB), were compared with those of a mixture of the same active ingredients, not complexed (denoted by MEB) and of a placebo composition without CN-MEB complexes or active ingredients MEB. The effects of MEB (not complexed) and CN-MEB (complexed) administered orally, topically, or in a combined oral+topical route, were clinically evaluated, according to non-invasive biophysical parameters. Topical application was performed on the face and/or the right arm of voluntary subjects. All subjects exhibited effects from photoaging. The tests performed, controlled by dermatologists to check skin conditions, are summarized in Table 1.

TABLE I
Exemplary administration mode for 2 days

| | ORAL | TOPICAL |
|---|---|---|
| 1 PLACEBO | Placebo 2 x day | Placebo 2 x day |
| 2 TOPICAL MEB | Placebo 2 x day | MEB 2 x day |
| 3 ORAL MEB | MEB 2 x day | Placebo 2 x day |
| 4 COMBINED MEB | MEB 2 x day | MEB 2 x day |
| 5 TOPICAL CN-MEB | Placebo 2 x day | CN-MEB 2 x day |
| 6 ORAL CN-MEB | CN-MEB 2 x day | Placebo 2 x day |
| 7 COMBINED CN-MEB | CN-MEB 2 x day | CN-MEB 2 x day |

MEB = melatonin, Vit. E, betaglucan; CN-MEB = chitin nanofibrils complexes, hyaluronic acid, melatonin, Vit. E, betaglucan

Test Products

Melatonin, Vit. E and betaglucan both non-complexed and complexed with chitin nanofibrils and hyaluronic acid, were employed in the form of oral hard capsules and as a topical nanoemulsion manufactured by MAVI SUD (Aprilia, Italy).

The CN complexes were obtained according to the protocol described herein in the detailed description. Every active capsule contained a mixture of the complexes with chitin nanofibrils (also denoted by CN in the description) in quantity of 1.6 mg for each active ingredient (in this case melatonin, Vit. E and betaglucan) individually complexed by 1.4 mg of chitin nanofibrils/hyaluronic acid dispersed in butylene glycol. The active emulsion used contained the MEB or CN-MEB complexes so as to obtain a final concentration of 2 μg/ml for any active ingredient.

Test subjects ingested one capsule twice a day (morning and evening) for oral administration, with meals. Topical administration was performed by applying the products on face, neck and right arm after cleansing these skin areas with a cleansing milk.

Combined administration was the same, but with half the dosage for each type of administration.

Measurement of skin hydration and superficial skin lipids.

Skin hydration and superficial skin lipids were evaluated by using the 3C System methodology described by Cardillo and Morganti “A fast non invasive method for skin hydration control” J. Appl. Cosmetol 12 11-13 1994. This instrument (Dermotech Italia Srl) has a separate probe for each of the test parameters. The probes on this computerized instrument collect up to 15 separate readings over a 25 second sampling period. On the days of laboratory evaluations, the skin was cleansed in the morning before measurements were taken and left undisturbed until after these measurements were completed. The topical test product was only applied after measurements were completed. These individual readings were taken on the area between the nose and cheek and were automatically averaged together. The resulting mean value was stored in the computer after standardization for environmental conditions (relative humidity: 50%, temperature: 22° C.). The probe employed in the 3C System for the measurement of skin hydration specifically assesses the total capacitance of the epidermis. The values, expressed in arbitrary units by the computer-controlled system, are automatically reported as a percent increase from baseline values measured within the 15 days prior to initiation of the study. All skin hydration measurements were taken under standardized conditions, according to what described in Pinnagoda J. Standardization of measurements. (1994) In: Eisner P, Berardesca E, and Maibach H, eds. Bioengineering and the skin: water and stratum corneum. Boca Raton: CRC Press; 59-65.

The probe employed in the 3C system for the measurement of superficial skin lipids employs a one square centimeter frosted plastic foil surface which becomes transparent in direct proportion to the amount of lipids present on the skin. The change in the light transmission of the foil is automatically recorded by the 3C system and converted to milligrams of lipid per square centimeter of skin surface. These converted values are automatically reported as a percent increase in superficial skin lipids from baseline values measured within the 15 days prior to initiation of the study by the computer-controlled system.

Skin Elasticity

Skin elasticity was assessed on the right forearm using a Dermaflex A instrument (Cortex Technology, Hadsund, Denmark) according to the method described in Gniadecka M, Serup J. (1995) (Suction chamber method for measurement of skin mechanical properties: the Dermaflex. In: Serup J, Jemec G, eds. Handbook of non-invasive methods and the skin. Boca Raton: CRC Press; 329-334). This instrument measures the extension of the skin in response to a suction vacuum induced above the skin test site with a 300 millibar vacuum; 20-second exposure period and 5 cycles per measurement. The relative elastic retraction (RER) was calculated from the equation described in the work by Gniadecka et al mentioned above. The values obtained were calculated as percent increase compared to baseline measured in the 15 days prior to the initiation of the study.

Skin Lipid Peroxidation

Values related to the degree of oxidation of skin lipids were determined by the method described in Ohkido M, Yoshino K, Matsuo I (1980) (Lipid peroxide of human skin. Curr Probl Dermatol 10:269-78). The amount of peroxides in skin lipids was measured in terms of the amount of malondialdehyde (MDA) generated in skin lipids following irradiation of the test site with a measured light exposure (5.6 erg/cm2/min for 2 minutes) from a high pressure UV light source (Osram 300 Watt lamp in the wavelength region of 240 and 320 nm) equipped with a monochronometer and a photodetector (Model IL700 International Light, Newbury, Mass., USA). Ten minutes after irradiation, skin lipids were extracted from the surface of the arm-skin by the cup method using two acetone extractions with a total volume of 10 ml. The extraction procedure and the MDA quantification are described in Ohkido 1980. In summary, an aliquot of the extracted lipids is added to sodium dodecyl sulfate in distilled water, adjusted to pH 4 with 20% acetic acid. Then, thiobarbituric acid is added to this medium and the entire mixture is heated to 95° C. for 60 minutes. After cooling to room temperature, n-butanol is added and the sample is centrifuged. The absorption of the n-butanol layer is then measured on a spectrophotometer at 532 nm. The amount of oxidized skin lipids is reported as nanograms of MDA per 100 mg of lipid.

Statistical Evaluations

All results (reported in the Figures) are presented as the mean value+1 standard deviation. The standard deviation values obtained in this study were similar to that obtained in Palombo P, Fabrizi G, Ruocco V, Ruocco, Flühr J, Roberts R, and Morganti P. (2007) (Beneficial long-term effects of combined oral/topical antioxidant treatment with carotenoids lutein and zeaxanthin on human skin: A double-blinded, placebo-controlled study in humans Skin. Pharmacol Physiol, 20:199-210). The baseline values were employed in the statistical evaluations where appropriate. Statistical evaluations were performed with the program GraphPad Prism® 4 (GraphPad Software Inc., San Diego, Calif., USA). All statistical evaluations were conducted as two-tailed analyses at a minimum of a 95% confidence (p<0.05) using a repeated measures ANOVA and a Tukey post-test to determine statistically significant differences in the results. The statistical comparisons employed were between each of the three MEB and CN-MEB treatments (oral, topical, or combination), and the placebo treatments at the same weeks of evaluation 4,8,12 in order to demonstrate the continued effect of the MEB treatments in comparison with CN-MEB also.

Clinical Evaluation

Assessment of skin photoaging.

In accordance with a previous study in Morganti P, Fabrizi G (1999) safety evaluation of phytosphingosine and ceramides of pharmaceutical grade. J. Appl. Cosmetol 17: 1-9, clinical evaluations were performed on day 1 (baseline) and at weeks 4, 8 and 12 (end of treatment). Skin examination of photoaging was evaluated by using a visual analogue score scale on: degree and appearance of fine wrinkles in the lateral-periorbital area, senile dryness, skin atrophy (thinning), black spots and teleangectasia in the whole face of the subjects. Measured scale scores were: 0 normal skin, 1-3 low presence of fine wrinkles, 4-6 moderate presence of wrinkling with some black spots, 7-9 high presence of wrinkles, black spots with telangectasia.

Results and Comments

The administration of MEB alone or complexed with chitin nanofibrils (CN-MEB), highlighted statistically significant results in all the evaluated parameters (p<0.05) showing the largest change with the combined topical+oral treatments (p<0.005). Moreover, the MEB-CN oral, topical and combined treatments resulted always statistically significant with all the parameters tested not only versus the placebo (p<0.005), but also versus all MEB combinations. FIGS. 1 and 2 show that, while topical skin hydration and skin surface lipids resulted statistically higher (p<0.05) in comparison with the oral administration for both MEB and CN-MEB, all values obtained with CN-MEB resulted higher in comparison with oral or topical MEB alone, with a further increase at week 12, given the supposed greater stability of the complexes of the invention compared to the non-complexed active ingredients. Skin elasticity data in FIG. 3 shows how the topical and oral CN-MEB values resulted statistically higher (p<0.05) than the correspondent MEB values for oral, topical and combined administrations. However, the results obtained for each of the weeks evaluated were not significantly different from each other, with exclusion of week 12 (p<0.05).

The same and statistically significant results were obtained for the reduction in skin lipid peroxidation, as measured by the amount of MDA formed throughout the study period in comparison to the placebo treatment. In this case as well, the combined oral+topical treatment has shown the greatest continuous decrease in lipid peroxidation, especially with the CN-MEB complexes. The decrease resulted statistically significant for all week periods evaluated (p<0.05) (FIG. 4). Clinical evaluation then confirmed the higher efficacy of the combined oral+topical administration (p<0.05), which further increased when MEB active ingredients were complexed with CN (p<0.05). Moreover, differently from biophysical testing, the clinical efficacy increases continuously from week 4 to week 12 (FIG. 5).

Accordingly, the studies reported above indicate that the administration of active ingredients complexed with chitin nanofibrils (CN) linked in turn to a suitable polymer negatively charged as described herein, and in particular contemporarily administered topically and by oral route, provides multiple advantageous effects in treatments of the skin or of epithelia analogously topically treatable.

### REFERENCES

Cardillo A, Morganti P. (1994) A fast non-invasive method for skin hydration control. J. Appl Cosmetol 12:11-13.
Gniadecka M, Serup J. (1995) Suction chamber method for measurement of skin mechanical properties: the Dermaflex. In: Serup J, Jemec G, eds. Handbook of non-invasive methods and the skin. Boca Raton: CRC Press; 329-334.
Morganti P, Fabrizi G (1999) safety evaluation of phytosphingosine and ceramides of pharmaceutical grade. J. Appl. Cosmetol 17: 1-9.
Ohkido M, Yoshino K, Matsuo I (1980) Lipid peroxide of human skin. Curr Probl Dermatol 10:269-78.
Palombo P, Fabrizi G, Ruocco V, Ruocco, Flühr J, Roberts R, and Morganti P. (2007) Beneficial long-term effects of combined oral/topical antioxidant treatment with carotenoids lutein and zeaxanthin on human skin: A double-blinded, placebo-controlled study in humans Skin. Pharmacol Physiol, 20:199-210.
Pinnagoda J. Standardization of measurements. (1994) In: Eisner P, Berardesca E, and Maibach H, eds. Bioengineering and the skin: water and stratum corneum. Boca Raton: CRC Press; 59-65.

1. Complexes of chitin nanofibrils, at least one negatively charged polymer, and one or more active ingredients, wherein the negatively charged polymer is selected from the group consisting of: hyaluronic acid, collagen, phospholipids, silicone polymers or oligomers, natural or synthetic peptides selected from the group comprising polyphenolic peptides, and polyglucosides.

2. The complexes according to claim 1, wherein said one or more active ingredients are selected from the group consisting of: hormones, immunostimulants, antioxidants, anti-inflammatory, antibacterial, antifungal, cicatrizing agents, vitamins, and oligominerals.

3. The complexes according to claim 2, wherein said one or more active ingredients are selected from the group consisting of: melatonin, phytoextrogens, ectoine, beta-glucan, carboxymethyl-betaglucan, zinc gluconate, lactate, picolinate, polyunsaturated fatty acids (PUFA), beta-carotene, lutein, zeaxanthin, lycopene, proanthocyanins, flavonoids, polyphenols, lipoic acid, vitamin A, vitamin C, vitamin E, tocotrienols, coenzyme Q10, creatine, nicotinamide, glycyrrhetic acid, phytosphyngosine, polyunsaturated fatty acids (PUFA), corticosteroids, zinc pyrithione and pyrithione olamine, ketoconazole, phytosphyngosine, chlorhexidrine gluconate, glycin, benzoyl peroxide, and silver.

4. The complexes according to claim 1, wherein said one or more active ingredients are selected from the group consisting of: melatonin, Vitamin E, and beta-glucan, or melatonin, lutein, beta-glucan, or melatonin, lipoic acid, ectoine, or melatonin, beta-carotene, beta-glucan, and the sole lutein.

5. The complexes according to claim 1, wherein said negatively charged molecule is hyaluronic acid.

6. A method of medical treatment using the complexes according to claim 1, comprising administering said complexes to a subject in need thereof.

7. The method according to claim 6, wherein a skin pathology of the subject is treated.

8. The method according to claim 6, wherein said complexes are administered by a route selected from the group consisting of: topical, oral, intradermal, subcutaneous, vaginal, ocular, nasal, recta, and combinations thereof.

9. The method according to claim 6, wherein skin chronoaging, skin photoaging, temporary or definitive skin changes, such as acne, oily or dry skin, keratosis, rosacea, sensitivity to light, skin spots from depigmentation or hyperpigmentation, xerosis and/or skin inflammation processes; allergic or autoimmune reactions, such as dermatoses and photodermatoses; anomalous skin cicatrization forms, such as formation of hypertrophic scars or keloids; skin atrophy states; loss of elasticity; and/or reducing the appearance of wrinkles, thin lines, stretch marks or cellulitis; forms of cutaneous hyperkeratoses, such as acne, eczema, lupus erythematosus, atopic dermatitis, psoriasis, allergic and/or contact dermatitis, hypersensitivity reactions, skin cancer, cutaneous melanoma, dryness of vaginal and/or ocular mucosae, forms of cataract and ocular macular degeneration are treated in a patient, wherein said complexes are administered to said patient in therapeutically effective doses.

10. A composition comprising the complexes according to claim 1, a suitable excipient, and optionally suitable adjuvants and/or additives.

11. The composition according to claim 10, wherein said composition is a pharmaceutical or cosmetic composition.

12. The composition according to claim 10, formulated for a topical, oral, intradermal, subcutaneous, vaginal, ocular, nasal, rectal administration or combinations thereof.

13. The composition according to claim 12, in the form of solution, colloidal solution, lotion, cream, macro, micro or nano emulsion, dispersion, gel, spray, foam, film, face mask, patches, bioactive fabrics or nonwoven tissues, tablet, hard or soft capsule, powder, granules, lyophilizate, syrup, or elixir.

14. A kit comprising aliquots of the composition according to claim 11 formulated for oral administration, and aliquots of the same composition formulated for topical administration for concomitant or sequential use thereof.

15. (canceled)

16. A method of preparation of complexes of chitin nanofibrils in association with at least one negatively charged polymer and one or more active ingredients according to claim 1, comprising:
a) preparing a first component by stirring from 5 min to 2 h a mixture in an aqueous medium of chitin nanofibrils and optionally one or more of said active ingredients;
b) preparing a second component by suspending in an aqueous medium at least one negatively charged polymer, and optionally one or more of said active ingredients; and
c) mixing the first and the second component for about 1 hour with obtainment of a precipitate of the complexes and a supernatant, provided that either component (a) or component (b) contain at least one active ingredient.

17. The method according to claim 16, wherein the amount of chitin in the first component ranges from 0.1% to 10% and the amount of negatively charged polymer in the second component ranges from 0.1% to 10%.

18. The method according to claim 16, further comprising separating the precipitate obtained at step (c) from the supernatant.

19. The method according to claim 16, further comprising subjecting the mixture or the precipitate obtained at step c) to pressure through a turbine, cylinders, thereby obtaining particles of micrometric or nanometric sizes.

20. The method according to claim 16, wherein at least one active ingredient is liposoluble and wherein the component containing it comprises a surfactant.

21. A method of preparation of pharmaceutical or cosmetic compositions containing chitin nanofibrils complexes in association with at least one negatively charged polymer and one or more active ingredients according to claim 1, comprising introducing said complexes into a suitable excipient and optionally further refining the mixture thus obtained.

22. The method according to claim 21, further comprising formulating the composition for topical, oral, ocular, nasal, rectal, or vaginal administration or for combined topical/oral administration, wherein said topical route comprises administration through biofabrics on nonwoven tissues or films.

23. A method of preparation of the kit according to claim 14, comprising introducing aliquots of a composition formulated for oral administration and aliquots of the same composition formulated for topical administration into a blister for their concomitant or sequential use.
